(19) United States
(12) Patent Application Publication
(10) Pub. No.: US 2003/0119156 A1
(43) Pub. Date: Jun. 26, 2003

(54) Biosynthesis of cyclic siloxanes

(76) Inventor: Nabil Yaqub Sakkab, Brussels (BE)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/320,877

(22) Filed: Dec. 17, 2002

Related U.S. Application Data
(60) Provisional application No. 60/342,715, filed on Dec. 20, 2001.

(51) Int. Cl. C12P 9/00

(52) U.S. Cl. 435/131

(57) ABSTRACT

Processes for making siloxanes, more particularly biosynthetic processes for making cyclic siloxanes are provided by the present invention.

### RELATED APPLICATIONS

This application claims priority to U.S. Provisional Application Serial No. 60/342,715 filed Dec. 20, 2001.

### FIELD OF THE INVENTION

The present invention relates to processes for making siloxanes, more particularly to biosynthetic processes for making siloxanes, especially cyclic siloxanes.

### BACKGROUND OF THE INVENTION

The industrial synthesis of polymeric silicones comprises passing methyl chloride through a fluidized bed of copper and silicon at high temperatures to produce a mixture of chlorosilanes which are subsequently hydrolyzed to yield mixtures of cyclic and linear silanol-terminated oligomers which can then be separated by distillation (Kirk-Othmer Encyclopedia of Chemical Technology, Volume 22, John Wiley & Sons, 1997 pp 84-90). The process runs at high temperatures and extremes of pH requiring significant energy input.

By contrast, synthesis of ordered silica structures in nature is known to occur at ambient pH's and temperatures apparently facilitated by organic components such as proteins and polysaccharides. Silica spicules isolated from the aquatic sponge Tethya aurantia have been shown to contain an axial protein filament termed a silicatein which is believed to be the protein scaffolding upon which the spicules are biosynthesized. Silicateins are composed of three very similar subunits: alpha (α) with a Molecular Weight of 29 kDa, beta (β) with a Molecular Weight of 28 kDa, and gamma (γ) with a Molecular Weight of 27 kDa. Recently, it has been shown that intact silicateins and the individual subunits are capable of promoting condensation of silica and organically modified siloxane polymers in vitro from the corresponding silicon alkoxides (K. Shimizu, J. Cha, G. D. Stucky, and D. E. Morse, Proc. Natl. Acad. Sci., USA 95, 6234-6238, 1998).

The alpha subunit of silicatein represents 70% of the slicatein filament and shows high homology to papain-like cystein protease, subfamily Cathepsin L. In the catalytic triad of the active center Histidine and Asparagine are conserved but Serine replaces Cysteine making the alpha subunit homologous to the subtilisin serine proteases.

Previous work (K. Shimizu, J. Cha, G. D. Stucky, and D. E. Morse, Proc. Natl. Acad. Sci., USA 95, 6234-6238, 1998), (J. N. Cha, K. Shimizu, Y. Zhou, S. C. Christiansen, B. F. Chmelka, G. D. Stucky, and D. E. Morse, Proc. Natl. Acad. Sci, USE 96 361-365, 1999), (J. N. Cha, G. D. Stucky, D. E. Morse, and T. J. Deming, Nature, Vol 403, pp 289-292, 2000) showed that the alpha subunit of slicatein is capable of promoting the condensation of tetraethoxysilane into polymeric siloxanes under relatively mild reaction conditions (room temperature, pH 6.8). Polycondensation of siloxane monomer is achieved via silicatein-mediated scaffolding and, likely, by silicatein-mediated catalysis of the polysiloxane formation. The silica is formed in layers on the underlying silicatein protein fiber. The scaffolding activity relates to the spatial distribution of hydroxyl groups on the silicatein protein, aligning the siloxane monomers in a favorable juxtaposition for the polycondensation. It is speculated that the catalytic activity resembles hydrolase's mechanism converting the slicon alkoxides to the corresponding silanol, known to condense spontaneously to polysiloxane (D. E. Morse, TIBTECH, Volume 17, June 1999, pp. 230-232).

Accordingly, it an object of the present invention to provide a process for the controlled synthesis of polymeric silicones by protein mediated condensation of the corresponding alkylalkoxysilane monomers in the presence of a solid particle having an average pore size sufficient to allow entry of polymeric silicones at the target size and below while rejecting larger polymers. By application of said process it has been found the polymeric silicones of a defined size can be synthesized under mild reaction conditions in high yields.

### SUMMARY OF THE INVENTION

The present invention fulfills the need described above by providing a process for making siloxanes, especially a biosynthetic process for making cyclic siloxanes and/or a controlled process for making cyclic siloxanes.

The present invention relates to a process for production of polymeric silicones of a defined size and Molecular Weight. Such process may utilize an organosilane monomer, a condensation catalyst for said organosilane monomer, a porous solid substrate wherein the pore size can be designed to fit only the polymeric silicones of the desired length and shorter, and a reacting solvent system that solubilizes the desired organosilane monomer, and all polymeric silicones of a size smaller than the target polymeric silicone such that the target polymeric silicone and any larger species are insoluble in said reacting solvent.

The present invention relates to a process for making polymeric silicones of defined size under mild reaction conditions. The process can utilize a protein catalyst to condense substituted organosilane monomers at temperatures from about 25 to about 40° C. in the presence of a solid particle having a pore size sufficient to allow silicone condensates of the target size and below to enter but excludes larger molecules.

It has been surprisingly found that the starting organosilane monomers have different solubilities than the polymeric silicone products allowing easy separation of the condensate products from the reactant stream.

More particularly, the invention relates to the use of silicatein protein subunits or modified subtilisin proteases attached to solid support particles having a defined pore geometry to synthesize polymeric silicones of a given size from organosilane monomers under mild reaction conditions.

It has now been surprisingly found that silicatein alpha can promote the condensation of alkylalkoxydesilanes under similarly mild conditions to generate the corresponding polymeric dialkyl siloxanes. However, in the absence of an appropriate template the reaction yields polymers with a wide range of size and Molecular Weight.

It is a further object of this invention to provide a process capable of synthesizing decamethylcyclicpentasiloxane in high yield from the dimethyldimethoxysilane (DMDMS) monomer under mild reaction conditions using Zeolites, Cyclodextrins, activated charcoal or Porous Starch particles with average pore sizes of 17 nanometers in combination with a protein catalyst. Such materials allow entry of condensates of the DMDMS-protein reaction up to and including decamethyl pentasiloxane. We have found that by retaining these materials in close proximity in a cavity of defined dimensions the equilibrium between the various silicone condensates can be significantly shifted to favor formation of the decamethylcyclicpentasiloxane species.

An aspect of the present invention is that a wild type and/or variant subtilisin protease can be used to promote the efficient condensation of organosilane monomers into polymeric silicones. Use of such enzymes has been found to significantly speed up the rate of polymeric silicone formation.

Another aspect of the present invention is to provide a process capable of synthesizing polymeric silicones in high yield under mild reaction conditions by linking a protein-based condensation catalyst to a porous solid support material having an average pore size sufficient to allow entry by the target polymeric silicone, and those silicones of smaller size, while rejecting larger condensates, is provided. The resulting supported catalyst can then be packed into a column through which reactant and solvent can be poured through the top and polymeric silicones collected at the bottom.

In one aspect of the present invention, a process for making a siloxane-containing material, is provided. The process comprises the steps of:

a) providing a silane, such as an organosilane, of the formula:

RₐSi-L_{b}

wherein R is independently —H or a hydrocarbon group, typically containing from about 1 to about 10 carbon atoms, more typically —CH₃, or —CH₂CH₃; and L is a leaving group, typically selected independently from -halide, —OH, —OCH₃ or —OCH₂CH₃; a and b selected such that the sum of a and b is 4; and

b) reacting the silane with a condensation catalyst (typically a protein) such that the leaving group is displaced from silane and at least one —Si—O—Si— bond is formed in the product of the reaction; and

c) optionally, recovering the product of the reaction from step b);

d) optionally, cyclizing the product of the reaction from step b) to produce a cyclic siloxane.

In another aspect of the present invention, a siloxane produced by the process according to the present invention, is provided.

Accordingly, the present invention provides a process for making siloxanes, more particularly a biosynthetic process for making cyclic siloxanes and siloxanes produced by such processes.

### DETAILED DESCRIPTION OF THE INVENTION

Silanes

The silanes, especially organosilanes, useful in the process of the present invention include silanes having the formula:

RₐSi-L_{b}

wherein R is independently —H or a hydrocarbon group, typically containing from about 1 to about 10 carbon atoms, more typically —CH₃, or —CH₂CH₃; and L is a leaving group, typically selected independently from -halide, —OH, —OCH₃ or —OCH₂CH₃; a and b selected such that the sum of a and b is 4.

In one embodiment, a and b are independently selected from 1 to 3. Accordingly, at least one leaving group (L) is present in the silane.

In another embodiment, at least one leaving group is present in the silane. In other words, b is 1 to 4.

In yet another embodiment, R is a hydrocarbon group containing from about 1 to about 4 carbon atoms.

In still another embodiment, at least one L is a halide, such as —Cl, —Br, and —I.

In still yet another embodiment, the silane is one in which a and b are each at least 1, and at least one R is —CH₃ or —CH₂CH₃ and at least one L is —OH or —OCH₃.

In even yet another embodiment, the silane is one in which at a is 2 and R is indepenendently —CH₃ or CH₂CH₃ and b is 2 and L is independently —OCH₃ or —OCH₂CH₃.

A nonlimiting example of a suitable silane is dimethyl dimethoxy silane (DDMS), which has the formula:

(CH₃)₂Si(OCH₃)₂.

Condensation Catalyst

The condensation catalyst for the processes of the present invention may be a protein. The condensation catalysts useful in the processes of the present invention include condensation catalysts that are capable of polymerizing Si-containing materials, especially to form —Si—O—Si—bonds.

Nonlimiting examples of suitable proteins include silicateins.

In one embodiment, the protein is a filamentous proteins isolated from the silica spicules of Tethya aurantia. Such proteins are comprised of three nearly identical subunits: alpha (α) of Molecular Weight 29 kDa, beta (β) with a Molecular Weight of 28 kDa, and gamma (γ) with a Molecular Weight of 27 kDa in a ratio of 12:6:1. The α subunit is preferred as it occurs in the highest concentration. The α protein may be isolated by traditional techniques or produced in high yield through the use of recombinant DNA technology using the cDNA sequence reported by Shimizu et al (Proc. Natl. Acad Sci USA Vol. 95 pp 6234-6238, 1998).

In another embodiment, the protein is a protease enzyme and/or variants thereof. Nonlimiting examples of suitable protease enzymes are the subtilisins which are obtained from particular strains of B. subtilis, B. licheniformis and B. amyloliquefaciens (subtilisin BPN and BPN), B. alcalophilus and B. lentus. Suitable Bacillus protease is Esperease® with maximum activity at pH 8-12, sold by Novozymes and described with its analogues in GB 1,243,784. Other suitable proteases include Alcalase®, Everlase® and Savinase® from Novozymes. Proteolytic enzymes also encompass modified bacterial serine proteases, such as those described in EP 251 446 (particularly pages 17, 24 and 98), referred to as “Protease B”, and in EP 199 404 which refers to a modified enzyme referred to as “Protease A”. Also suitable is the enzyme called “Protease C”, which is a variant of an alkaline serine protease from Bacillus (WO 91/06637). A preferred protease referred to as “Protease D” is a carbonyl hydrolase variant having an amino acid sequence not found in nature, described in WO95/10591 and WO95/10592. Preferred proteases are multiply-substituted protease variants comprising a substitution of an amino acid residue at positions corresponding to positions 103 and 76, there is also a substitution of an amino acid residue at one or more amino acid residue positions other than amino acid residue positions corresponding to positions 27, 99, 101, 104, 107, 109, 123, 128, 166, 204, 206, 210, 216, 217, 218, 222, 260, 265 or 274 of Bacillus amyloliquefaciens subtilisin. WO 99/20723, WO99/20726, WO99/20727, WO99/20769, WO99/20770 and WO99/20771 describe also suitable proteases, wherein preferred variants have the amino acid substitution set 101/103/104/159/232/236/245/248/252, more preferably 101G/103A/104I/159D/232V/236H/245R/248D/252K according to the BPN′ numbering.

In still another embodiment, the protein is a subtilisin protease variant with specific changes designed to enhance the condensation of the organosilane and remain stable under the conditions of the claimed process. Such variants can be generated by a number of standard methods known in the art. Preferred is a process wherein random variants are produced through PCR mutagenesis of the entire gene, said mutant genes are inserted into a suitable bacillus expression system, and variant proteins excreted extracellularly. This method is particularly effective when coupled with a high throughput screening technique that selects enzymes based on activity and stability in the solvent systems of choice for the claimed process.

Alternatively, the condensation catalyst may be a peptide. Nonlimiting examples of suitable peptides are peptides with a high affinity for binding and condensing the organosilane monomers. Such peptides may be prepared using standard methods known in the art. A preferred method is generation of a large, randomly mutated library of peptides via phage display techniques followed by screening for high binding peptides in a suitable high throughput assay. Such an approach yields peptides with high binding affinity for the organosilane. At sufficient concentrations in the solvents of choice the combination of a binding peptide with an organosilane leads to condensation of the monomer to polymeric silicones.

Substrate

The reaction of the silane with the condensation catalysts may occur within a substrate and/or porous support so that the desired reaction product is obtained.

One way that the size of the reaction product (i.e., siloxanes) can be controlled is by physically defining the environment in which the reaction of the silane with the protein occurs.

Any non-reactive substrate, “non-reactive substrate” as used herein means any substrate made of a material that will not react and/or interfere with the reaction of the silane with the protein. For example, a subtrate that contains no free silane groups.

Nonlimiting examples of such non-reactive substrates are solid structures that encompass pores and/or holes and/or indentations of such a size to hold the desired cyclic siloxane to be produced can be used. Suitable soild structures can be selected for use in the process of the present invention based upon their pore and/or hole and/or indentation sizes. For example, once a desired cyclic siloxane has been identified, the size of such cyclic siloxane can be calculated either by actual measurement of the desired cyclic siloxane to be produced or by theoretical measurement using any number of computer programs and/or other theoretical means for measuring the desired cyclic siloxane.

The porous support is selected from materials that are inert to condensation or reaction with the organosilane monomer and resulting polymer silicone and have average pore sizes sufficient to allow entry of all polymeric silicones of the target size and smaller. Examples of such materials include Zeolites, cyclodextrins, porous starches, dextrose beads such as Sphadex, cross-linked polymers of acrylamide, Sphearose, and activated carbon. Certain modified celluloses such as DEAE-cellulose are also suitable.

In order to be an object of the present invention the porous support must satisfy two criteria. First, it must remain inert to the organosilane monomer and the resulting polymeric silicone condensates under reaction conditions. Mixing the porous support of interest with the monomer in an appropriate solvent and allowing the mixture to stand at 40° C. for several hours can test reactivity with the organosilane monomer. Analysis of the system by gas chromatography, HPLC, ion-chromatography, mass spectroscopy or any other suitable analytical method should not indicate the presence of appreciable quantities of condensed silicones. Zeolites, porous carbon supports, porous starches, cyclodextrins, porous cellulose beads and cross-linked polymers of acrylamide are all suitable supports for the claimed process.

The second criterion that must be satisfied in order for a porous support to be considered an object of the present invention is the average size of the pores. Appropriate pore size is defined by the desired size of the polymeric silicone. Using molecular modeling programs such as Spartan™ average geometries for the polymeric silicones of interest can be determined. Calculating molecular dimensions of the target polymeric silicone as well as the expected condensates of smaller and larger size allows one to define the range of pore size required to achieve the polymeric silicone of interest according to the general formula:

Avg. Pore Size≦Dmax

where Dmax is the calculated molecular diameter on the longest axis of the polymeric silicone of interest. All non-reactive substrates having a pore size at or below the calculated maximum diameter for the polymeric silicone of interest are acceptable.

Experimentally, the pore size of solid substrates can be determined by any number of methods known in the art. As a first approximation electron microscopy may be used to get a general idea of surface pore size. A preferred method is the generation of a BET-Nitrogen absorption isotherm from which average pore size can be calculated.

The substrate and/or porous materials can be used in any form. For example, by packing into a column and flowing the reactants over it.

The basic idea is to make D5 from the dimethlydimethoxy silane (DMDMS) monomer using silicatein as the catalyst to condense the monomers. We speculate that DMDMS is sparingly soluble in water and likely needs to be kept near neutral pH to avoid acid or base catalyzed polymerization. We run the reaction in a two phase system. There is an aqueous phase that contains the silicatein and the monomer. As the silicatein condenses the monomer the reaction products very quickly separate from the aqueous phase because they are water insoluble. As they separate you pass them over a fixed bed reactor that contains the silicatein immobilized on a support (lets say a non-silica support to avoid solubilization) and, at the same time, pass an aqueous stream of DMDMS so that the reaction can continue until you get decamethylpentasilane formed which can then spontaneously close to form D5. This last step might be facilitate by a template that holds the material in residence long enough for cyclization to occur.

Some variations of this idea are to immobilize the silicatein on the surface of a template that will only accomodate molecules of size of D5 or smaller. Nothing bigger than D5 can form in the template. In this execution you would pass an aqueous solution of DMDMS over the immobilized silicatein and collect D5 out the other end.

Another thought was to engineer BPN′ to replace the silicatein. Since BPN′ already has a hydrophobic binding pocket and two of the three amino acids in the active site homologous to silicatein we believe it would be straight forward to engineer the enzyme to do the catalytic chemistry. If such an enzyme could be made to catalyze condensation of DMDMS in aqueous media likely it could be made to hydrolyze the Si-O-Si bonds in non-aqueous media. That would then allow you to convert D3, D4 into D5 by adding enzyme plus DMDMS. Alternatively, you could hydrolyze higher cyclics like D6 and D7 down to the target chain length, combine with the molecular template, and form more D5. So this approach would be one way to enrich a product stream in D5. The engineering of BPN′ could be done either through site directed mutagenesis or by random mutagenesis directed evolution.

On the molecular template, we had a couple ideas. One was to use cyclodextrins since high concentrations of hydroxyl groups appear to be required in order to get templating. Another thought was to screen peptide libraries until we found peptides that only bound D5 and use them as templates.

What is claimed is:

1. A process for producing a polymeric silicone of defined length comprised of:
a. a organosilane monomer;
b. a condensation catalyst for said monomer;
c. a porous substrate wherein the pore size is designed to fit only the polymeric silicones of the desired length and shorter;
d. a reacting solvent system that solubilizes the desired organosilane monomer, and all polymeric silicones of a size smaller than the target polymeric silicone such that the target polymeric silicone and any larger species are insoluble in said reacting solvent;
e. a process for recovering the target polymeric silicone.

2. A process according to claim 1 wherein the condensation catalyst is attached or adsorbed to the porous substrate.

3. A process according to claim 1 wherein the condensation catalyst is comprised of a protein.

4. A process according to claim 3 wherein the protein is derived from the silica spicules of Tethya aurantia.

5. A process according to claim 3 wherein the protein is the alpha subunit of the protein filament derived from the silica spicules of Tethya aurantia with a molecular weight of about 29 kDa.

6. A process according to claim 3 wherein the protein is a variant form of the alpha subunit of the protein filament derived from the silica spicules of Tethya aurantia wherein the protein has been modified to allow attachment to a substrate.

7. A process according to claim 3 wherein the protein is a variant form of the alpha subunit of the protein filament derived from the silica spicules of Tethya aurantia wherein the protein has been modified for improved stability.

8. A process according to claim 3 wherein the protein is a variant form of the alpha subunit of the protein filament derived from the silica spicules of Tethya aurantia wherein the protein has been modified to improve the rate of condensation of organosilane monomer.

9. A process according to claim 3 wherein the protein is the beta subunit of the protein filament derived from the silica spicules of Tethya aurantia with a molecular weight of about 28 kDa.

10. A process according to claim 3 wherein the protein is a variant form of the beta subunit of the protein filament derived from the silica spicules of Tethya aurantia wherein the protein has been modified to allow attachment to a substrate.

11. A process according to claim 3 wherein the protein is a variant form of the beta subunit of the protein filament derived from the silica spicules of Tethya aurantia wherein the protein has been modified for improved stability.

12. A process according to claim 3 wherein the protein is a variant form of the beta subunit of the protein filament derived from the silica spicules of Tethya aurantia wherein the protein has been modified to improve the rate of condensation of organosilane monomer.

13. A process according to claim 3 wherein the protein is the gamma subunit of the protein filament derived from the silica spicules of Tethya aurantia and with a molecular weight of about 27 kDa.

14. A process according to claim 3 wherein the protein is a variant form of the gamma subunit of the protein filament derived from the silica spicules of Tethya aurantia wherein the protein has been modified to allow attachment to a substrate.

15. A process according to claim 3 wherein the protein is a variant form of the gamma subunit of the protein filament derived from the silica spicules of Tethya aurantia wherein the protein has been modified for improved stability.

16. A process according to claim 3 wherein the protein is a variant form of the gamma subunit of the protein filament derived from the silica spicules of Tethya aurantia wherein the protein has been modified to improve the rate of condensation of organosilane monomer.

17. A process according to claim 3 wherein the protein is an enzyme.

18. A process according to claim 17 wherein the enzyme is a native or mutant Subtilisin protease.

19. A process according to claim 17 wherein the enzyme is a native or mutant Cysteine protease.

20. A process according to claim 3 wherein the protein is a peptide obtained from screening a diverse peptide library.

21. A process according to claim 1 wherein the condensation catalyst and porous substrate are packed in a column that allows the reacting solvent system and organosilane monomer to be added at the top and the target polymeric silicone compound to be recovered at the bottom.

22. A process for producing a polymeric silicone of defined length comprised of:
a. the alpha subunit of the protein filament derived from the silica spicules of Tethya aurantia with a molecular weight of about 29 kDa;
b. a porous Sodium/Aluminum Zeolite wherein the pore size is designed to fit only the polymeric silicones of the desired length and shorter;
c. a silicone-based solvent system that solubilizes organosilane monomers, and all polymeric silicones of a size smaller than the target polysiloxane such that the target polymeric silicone and any larger species are insoluble in said silicone solvent;
d. a process for recovering the target polymeric silicone.

23. A process according to claim 22 wherein the alpha subunit protein is chemically or physically attached to the Zeolite.

24. A process according to claim 22 wherein the Zeolite is replaced by a cyclodextrin having a pore size sufficient to accommodate only the polymeric silicones of the desired length and shorter.

25. A process according to claim 22 wherein the Zeolite is replaced by activated carbon wherein the pore size is sufficient to accommodate only the polymeric silicones of the desired length and shorter.

26. A process according to claim 22 wherein the Zeolite is replaced by a porous starch particle wherein the pore size is sufficient to accommodate only the polymeric silicones of the desired length and shorter.

27. The process according to claim 22 wherein the alpha subunit protein is replaced by a wild type or variant protease enzyme.

28. The process according to claim 27 wherein the enzyme is chemically or physically attached to the Zeolite.

29. A process for making a siloxane, the process comprising the steps of
a) providing a silane of the formula:
RₐSi-L_{b}
wherein R is a hydrocarbon group; L is a leaving group; the sum of a and b is 4;
b) reacting the silane with a condensation catalyst such that the leaving group is displaced from the silane and at least one —Si—O—Si— bond is formed in the product of the reaction; and
c) optionally, recovering the product of the reaction from step b);
d) optionally, cyclizing the product of the reaction from step b) to produce a cyclic siloxane.
